(19) United States
(12) Patent Application Publication
(10) Pub. No.: US 2026/0272363 A1
(43) Pub. Date: Sep. 17, 2026

(54) Systems and Methods for Brain-Based Detection of Late Talking

(71) Applicant: University of Washington, Seattle, WA (US)

(72) Inventor: Bonnie Lau, Seattle, WA (US)

(73) Assignee: University of Washington, Seattle, WA (US)

(21) Appl. No.: 19/566,650

(22) Filed: Mar. 13, 2026

Related U.S. Application Data
(60) Provisional application No. 63/772,392, filed on Mar. 14, 2025.

(51) Int. Cl. A61B 5/38; A61B 5/00

(52) U.S. Cl. CPC A61B 5/38; A61B 5/7267; A61B 5/7275; A61B 2503/04

(57) ABSTRACT

Systems and methods for diagnostic assessment of language development in accordance with embodiments of the invention are illustrated. One embodiment includes a method for assessing language development. The method includes steps for obtaining a set of low-level neurological measurements based on a first set of stimuli, wherein the set of low-level neurological measurements includes an onset response latency measurement, obtaining a set of high-level neurological measurements based on a second set of stimuli, wherein the set of high-level neurological measurements are based on a temporal response function (TRF) method, obtaining a set of developmental measurements based on at least a set of behavioral screening assessments, and computing a predicted developmental trajectory based on a combination of the set of low-level neurological measurements, the set of high-level neurological measurements, and the set of developmental measurements.

### CROSS-REFERENCE TO RELATED APPLICATIONS

The current application claims the benefit of and priority under 35 U.S.C. § 119(e) to U.S. Provisional Patent Application No. 63/772,392 entitled “Systems and Methods for Brain-Based Detection of Late Talking” filed Mar. 14, 2025. The disclosure of U.S. Provisional Patent Application No. 63/772,392 is hereby incorporated by reference in its entirety for all purposes.

### STATEMENT OF FEDERAL SUPPORT

This invention was made with government support under Grant Nos. R00DC016640 and R01DC022585, awarded by the National Institutes of Health. The government has certain rights in the invention.

### FIELD OF THE INVENTION

The present invention generally relates to diagnostic assessment. More particularly, this specification is directed to diagnostic assessment of language development and disorder in young children.

### BACKGROUND

Delayed language emergence in the absence of other cognitive or developmental concerns occurs in approximately 10-20% of children, often referred to as “late talkers”. This condition often co-occurs with neurodevelopmental disorders, such as autism spectrum disorder or hearing impairment, that are likely to affect these children throughout their life. Nevertheless, starting intervention and treatment as early as possible is shown to lead to more optimal language and learning outcomes.

### SUMMARY OF THE INVENTION

Systems and methods for diagnostic assessment of language development in accordance with embodiments of the invention are illustrated. One embodiment includes a method for assessing language development. The method includes steps for obtaining a set of low-level neurological measurements based on a first set of stimuli, wherein the set of low-level neurological measurements includes an onset response latency measurement, obtaining a set of high-level neurological measurements based on a second set of stimuli, wherein the set of high-level neurological measurements are based on a temporal response function (TRF) method, obtaining a set of developmental measurements based on at least a set of behavioral screening assessments, and computing a predicted developmental trajectory based on a combination of the set of low-level neurological measurements, the set of high-level neurological measurements, and the set of developmental measurements.

In a further embodiment, the first set of stimuli includes stimuli with a characteristic selected from the group consisting of a long duration, a long interstimulus interval (ISI), and an increased sound level to ensure the most optimal neural response.

In still another embodiment, the set of low-level neurological measurements includes at least one selected from the group consisting of onset response latency, onset response amplitude, acoustic change response (ACR) latency, ACR amplitude, mismatch negativity, and hemispheric laterality.

In a still further embodiment, the set of high-level neurological measurements are based on a MTF.

In yet another embodiment, obtaining the set of high-level neurological measurements comprises decoding neurological signals to reconstruct a stimulus envelope using a trained machine learning model, and evaluating the reconstruction success for the stimulus envelope.

In a yet further embodiment, the set of low-level neurological measurements, the set of high-level neurological measurements, and the set of developmental measurements are for a particular subject, wherein the machine learning model is trained on data for the particular subject.

In another additional embodiment, the first and second sets of stimuli includes at least one selected from the group consisting of speech stimuli and visual stimuli.

In a further additional embodiment, the set of behavioral screening assessments includes at least one selected from the group consisting of the Parental Stress Scale, the Communication and Symbolic Behavior Scales Infant-Toddler Checklist, the Mullen Scales of Early Learning, the Vineland Adaptive Behavior Scales, the MacArthur-Bates Communicative Development Inventories Words and Gestures, the MacArthur-Bates Communicative Development Inventories Words and Sentences, and the Bayley Scales of Infant and Toddler Development.

In another embodiment again, at least one of the first set of neurological measurements or the second set of neurological measurements is based on an electroencephalogram (EEG) response to the first or second sets of stimuli.

In a further embodiment again, the first set of neurological measurements includes auditory evoked potentials.

In still yet another embodiment, the predicted developmental trajectory is derived from a mean growth trajectory determined based on multilevel linear regression modeling from a large sample of infants.

One embodiment includes a non-transitory machine readable medium containing program instructions that are executable by a set of one or more processors to perform a method comprising obtaining a set of low-level neurological measurements based on a first set of stimuli, wherein the set of low-level neurological measurements includes an onset response latency measurement, obtaining a set of high-level neurological measurements based on a second set of stimuli, wherein the set of high-level neurological measurements are based on a temporal response function (TRF) method, obtaining a set of developmental measurements based on at least a set of behavioral screening assessments, and computing a predicted developmental trajectory based on a combination of the set of low-level neurological measurements, the set of high-level neurological measurements, and the set of developmental measurements.

One embodiment includes a system for assessing language development, the system comprising a processor, a non-transitory machine readable medium containing program instructions that are executable by a set of one or more processors to perform a method comprising obtaining a set of low-level neurological measurements based on a first set of stimuli, wherein the set of low-level neurological measurements includes an onset response latency measurement, obtaining a set of high-level neurological measurements based on a second set of stimuli, wherein the set of high-level neurological measurements are based on a temporal response function (TRF) method, obtaining a set of developmental measurements based on at least a set of behavioral screening assessments, and computing a predicted developmental trajectory based on a combination of the set of low-level neurological measurements, the set of high-level neurological measurements, and the set of developmental measurements.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which form a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 conceptually illustrates a predictive diagnostic process for evaluating likelihood of Late Language Emergence (LLE) in accordance with some embodiments of the invention.

FIG. 2 illustrates a representation of the dispersion of onset latencies across different stages of infant development.

FIG. 3 depicts a representation of the association between language skills and onset latencies for infants.

FIG. 4 illustrates a representation of onset and acoustic change responses to speech averaged across various infants at the same stage of development.

FIG. 5 conceptually illustrates a process for reconstruction of stimulus envelopes in accordance with certain embodiments of the invention.

FIGS. 6A-6B illustrate representations evaluating the accuracy of speech envelopes reconstructed in accordance with numerous embodiments of the invention.

FIG. 7 illustrates a representation of the variability of various behavioral measures obtained in accordance with several embodiments of the invention.

FIGS. 8-9 conceptually illustrate an LLE detection system implemented in accordance with multiple embodiments of the invention.

### DETAILED DESCRIPTION

Systems and methods for facilitating prognostic evaluation of early-childhood language faculties are illustrated below. The identification of infants at a higher likelihood of language delay is especially valuable due to enabling early intervention. This benefit has been reported across diagnostic categories: for children with mild to profound hearing loss, age of enrollment into early intervention may have a dramatic impact on vocabulary scores; further, autistic children who start intervention earlier also demonstrate better outcomes. However, early identification is an especially challenging endeavor, as language screening and assessment is generally conducted after 18 to 24 months of age. One of the challenges with early screening is that children usually do not produce their first words until the end of the first year. Nevertheless, infants begin responding to sound in utero and show sophisticated speech discrimination from birth.

Systems and methods in accordance with many embodiments of the invention can utilize a combination of low-level measurements, high-level measurements, and/or developmental measurements to serve as prognostic predictors of future language capabilities, allowing for language screening as early as the first year of life. Processes in accordance with various embodiments of the invention may enable interventions and support of children with language difficulties during a period of maximum neuroplasticity, optimizing their eventual development.

### A. LLE Detection Processes

Systems and methods in accordance with many embodiments of the invention may evaluate a combination of low-level measurements (e.g., onset response latency/amplitude, mismatch negativity, hemispheric laterality, etc.), high-level measurements (e.g., speech envelope reconstruction accuracy, cortical tracking correlation, etc.), and/or developmental measurements (e.g., behavioral screening assessments, personal background attributes, etc.) to establish predictive diagnostics of late language emergence (LLE).

Measures in accordance with some embodiments of the invention may be based on brain responses to stimuli (e.g., speech, sounds, visual, etc.) and/or language assessments, including (but not limited to) norm-referenced standardized measures, language samples, and/or parent-reports of language and general development. In various embodiments, neurological measurements may be directed to high and low-level auditory brain responses including (but not limited to) latency estimates of the onset response to speech (e.g., auditory evoked potentials) and cortical tracking of the speech envelope. Although many of the examples described herein describe speech envelopes, one skilled in the art will recognize that similar systems and methods can be used with different stimuli envelopes without departing from this invention.

In various embodiments, LLE detection processes may account for developmental measurements based on personal background attributes, including (but not limited to) sex, social economic status, and/or maternal education. Behavioral analyses implemented in accordance with various embodiments may take the form of cross-domain assays of development including but not limited to auditory, language, cognitive, motor, and adaptive behaviors. Personal background attributes incorporated in accordance with several embodiments may include but are not limited to demographics, genetic features, and environmental indicators.

LLE detection processes in accordance with several embodiments of the invention may, additionally or alternatively, account for some or all of the above measures for infants or children at various ages. In accordance with a number of embodiments of the invention, language outcomes may be characterized by obtaining comprehensive brain-behavior trajectories for measurements across several (e.g., the first two post-birth) years.

Therefore, factors including but not limited to neurological measurements, behavioral analyses (e.g., quantitative assessments), and/or personal background factors may be used to: determine individual risk values, representative of likelihood of LLE; and/or reveal replicable markers of language delay across individuals.

a. Auditory Brain Responses

Systems and methods for LLE detection in accordance with many embodiments of the invention use auditory brain responses recorded in subject infancy to predict later language outcomes. In a variety of embodiments, LLE detection utilizes electroencephalography (EEG) data, to longitudinally track specific elements including (but not limited to) the latency of onset responses to speech and cortical tracking of speech envelopes across subjects. EEG allows for the recording of robust, temporally precise neural signals in infants from birth and has the potential for clinical application.

Although many of the examples described herein describe the use of data from EEGs, one skilled in the art will recognize that other types of neurological data can be used, including (but not limited to) Magnetic Resonance Imaging (MRI) scans, Computed Tomography (CT) scans, Positron Emission Tomography (PET) scans, Electromyography (EMG) scans, Magnetoencephalography (MEG) scans, and/or electrocorticography (ECoG) scans, without departing from this invention.

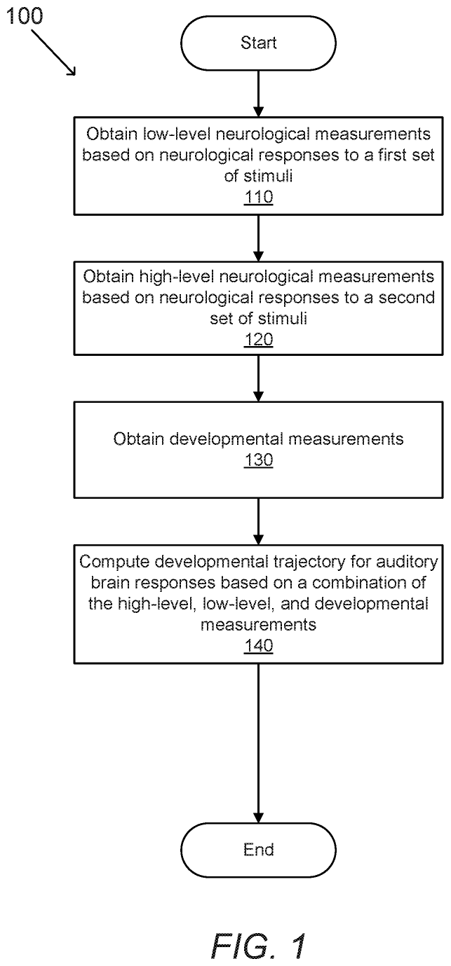

A process for language outcome predictions derived in accordance with many embodiments of the invention is illustrated in FIG. 1. In many embodiments of the invention, process 100 may be performed by a device that interacts with other elements, such as (but not limited to) EEG machines, databases, stimuli sources, etc.

Process 100 obtains (110) low-level neurological measurements based on neurological responses to a first set of stimuli. In several embodiments, low-level measurements may capture the integrity and maturation of the auditory pathway from the auditory nerve through the brainstem to the auditory cortex, providing an objective measure of neural transmission efficiency that may serve as an early indicator of language development trajectory. Low-level measurements in accordance with numerous embodiments of the invention include (but are not limited to) onset responses latency/amplitude, acoustic change response (ACR) latency/amplitude, mismatch negativity, and hemispheric laterality. In many embodiments, low-level measurements may be based on auditory evoked potentials (AEPs), which are described in greater detail below.

In a number of embodiments, the first set of speech stimuli is intended to prioritize low-level neurological function. In various embodiments of the invention, the first set of speech stimuli can include (but is not limited to) stimuli with a long duration, a long interstimulus interval (ISI), and/or an increased sound level to ensure the most optimal neural response.

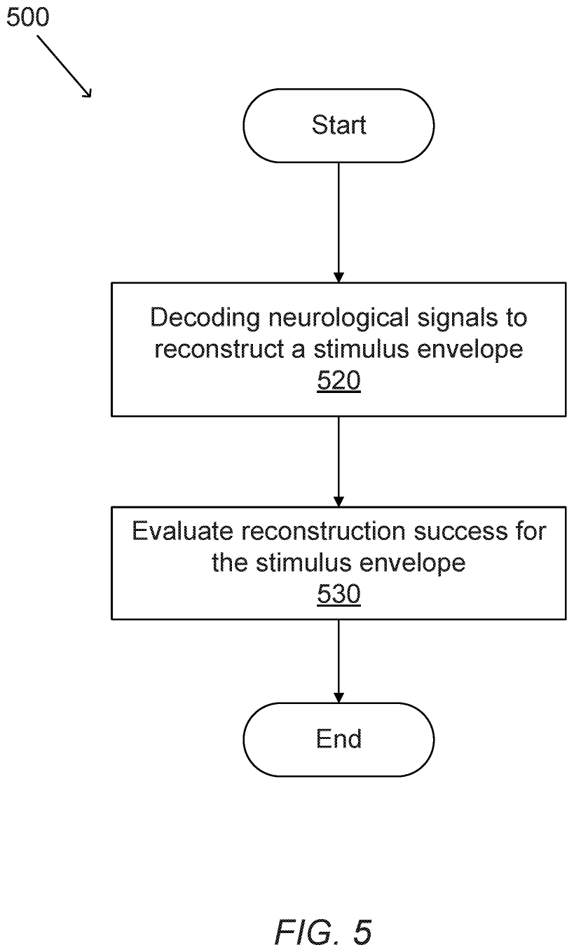

Process 100 obtains (120) high-level neurological measurements based on neurological responses to a second set of stimuli. In many embodiments, high-level neurological measurements may quantify the degree to which neural activity in auditory cortical regions synchronizes with the temporal dynamics of the speech signal, thereby providing an index of how effectively the infant brain processes continuous speech input. High-level measurements in accordance with certain embodiments of the invention are based on cortical tracking of the speech envelope. In several embodiments, high-level measurements can be computed by measuring the accuracy of speech envelope reconstruction. Speech envelope reconstruction is described in greater detail below (see e.g., FIG. 5).

In some embodiments, the second set of speech stimuli is intended to prioritize high-level neurological function (e.g., cortical processing). In numerous embodiments, the second set of speech stimuli can include (but is not limited to) stimuli that prioritizes real-world relevance by presenting naturalistic, continuous, infant-directed speech (IDS).

Obtaining neurological measurements in accordance with some embodiments of the invention can include providing stimuli, detecting responses to the stimuli, receiving recorded responses, etc. In numerous embodiments, stimuli can include (but are not limited to) sounds, speech, visuals, etc.

Process 100 obtains (130) developmental measures. As language development is complex, effective language screening in infancy may require a combination of brain and behavioral measures, interpreted within the context of demographic and environmental variables known to be related to language development. Developmental measures in accordance with certain embodiments of the invention are based on behavioral screening assessments (e.g., the Mullen Scales of Early Learning) and/or personal background attributes for the subjects. Personal background attributes may include, but are not limited to, environmental variables known to be related to language development like sex, socioeconomic status, and maternal education.

Process 100 computes (140) a developmental trajectory based on a combination of the high-level, low-level, and developmental measurements. In a variety of embodiments, computing a developmental trajectory includes estimating one or more predictive diagnostic scores based on the low-level, high-level, and developmental measures. In various embodiments of the invention, the predictive diagnostic score may be numerical and reflect a likelihood of LLE for individual subjects. Alternatively, or conjunctively, computing developmental trajectories in accordance with several embodiments of the invention is based on a rate of change in measurements over a period of time (e.g., between 6 and 11 months). In several embodiments, developmental trajectories may be computed based on a weighted combination of high-level, low-level, and developmental measurements. Developmental trajectories in accordance with several embodiments of the invention can utilize parallel latent growth modeling within a structural equation modeling framework. Growth trajectory parameter estimates can be used to predict clinically relevant language outcomes.

While specific processes for diagnosing language faculties are described above, any of a variety of processes can be utilized to provide predictions as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted. Although the above embodiments of the invention are described in reference to association with EEG measurements, the techniques disclosed herein may be used in any type of neurological measurements.

i. Auditory Evoked Potentials

Auditory evoked potentials (AEPs) are an objective brain measure that holds potential as predictors of later language outcomes. AEPs measure sound encoding and transmission throughout the entire auditory pathway—from the auditory nerve and brainstem through to the auditory cortex. The latency of AEP components may therefore operate as a parameter to assess maturation of the neural pathway, with newborns showing increased latencies compared to adults.

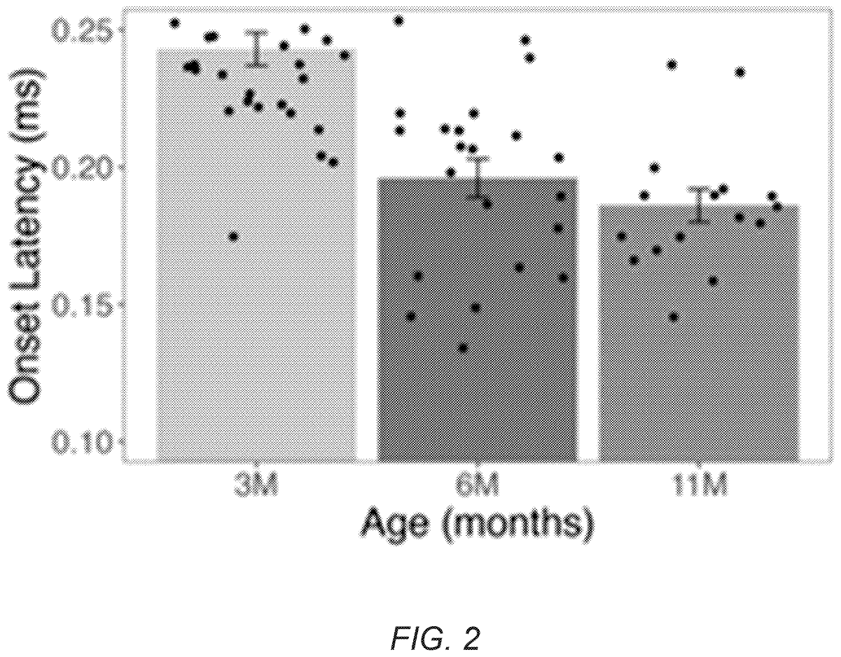

FIG. 2 depicts a representation of onset latencies (AEPs) at various ages. In particular, the graph of FIG. 2 shows the onset latencies of infants at 3 months (n=27), 6 months (n=22), and 11 months (n=16). The graph suggests a drastic decrease in the onset latencies over time, from around 0.24 milliseconds at 3 months, to 0.19 at 6 months, to 0.18 at 11 months. Decreases in AEP latencies with age are thought to reflect myelination, synaptic development, and improvements in auditory nerve synchrony, phase locking, and adaptation. As such, the developmental trajectory of auditory brain responses to speech may parallel trajectories of language development, suggesting that such AEPs can serve as a prognostic biomarker of late language emergence.

In evaluating AEPs, stimuli including but not limited to clicks, pure tones, synthetic speech sounds, and native/non-native speech contrasts may be implemented in accordance with various embodiments of the invention. In doing so, auditory brain responses may be recorded using AEP parameters including but not limited to the amplitude and latency of AEP peaks, mismatch negativity, and age-related morphological changes to the evoked neural response. Despite variability in stimuli and the parameters of interest, all of the above are predictive of later language outcomes.

The recording of neurophysiological responses in infants may be affected by low-level neural immaturity such as a lengthened auditory nerve refractory period. Given the extremely young age of some infants (e.g., 3 months), systems in accordance with some embodiments may be configured to present stimuli in modes including but not limited to a long duration, a long interstimulus interval (ISI), and an increased sound level to ensure the most optimal neural response. For example, a given trial can include a 2 second stimulus with a syllable change at 1 second. This stimulus may be followed by an ISI that is jittered randomly (e.g., between 1900 and 2100 ms) to maintain a more natural stimulus despite the long duration and to enable tracking of the neural response through both signal onset and the acoustic change. In various embodiments, due to the elevated absolute thresholds of infants (e.g., due to higher external ear resonant frequency, differences in the shape of the external ear transfer function, and higher middle ear impedance), the stimuli may be projected at an increased level compared to adults (e.g., 75 decibel sound pressure level—dB SPL)—for instance, through a loudspeaker in a sound-attenuated booth, calibrated with a sound level meter at the location of the participant's head.

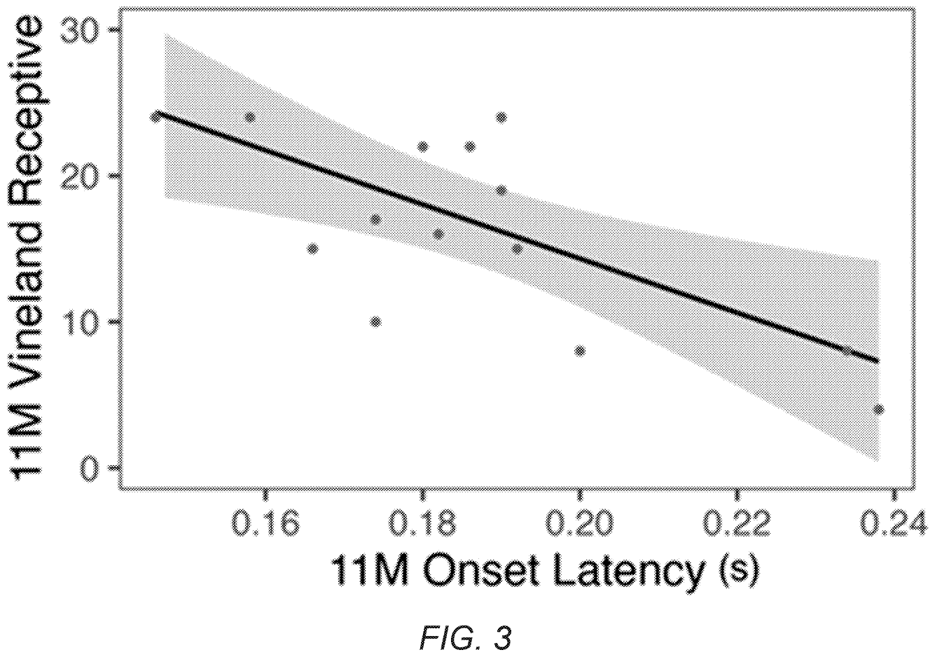

A representation of the association between language skills and latencies is illustrated in FIG. 3. The representation compares raw scores of a behavioral assessment (Vineland Adaptive Behavior Scales-Third Edition) and a measurement of onset latency at 11 months. Preliminary data shows that AEP latencies decrease over the first year of life and that longer latencies are observed at all infant timepoints than for adults. As mentioned above, decreased latencies are thought to reflect myelination, synaptic development, and maturation of auditory nerve synchrony and adaptation. Preliminary data on the subject demonstrates that there is sufficient variability in response latency to support the interpretation that it is a reliable prognostic predictor of late language emergence. Furthermore, by 11 months of age, the emergence of a relationship between receptive language skills and response latency is more firmly suggested.

In additional or alternative to onset latency, systems in accordance with some embodiments may incorporate additional measures that can be extracted from EEG recordings of onset responses. This may include, but is not limited to, the amplitude of the onset response, the latency and amplitude of the acoustic change response (ACR), and/or hemispheric laterality.

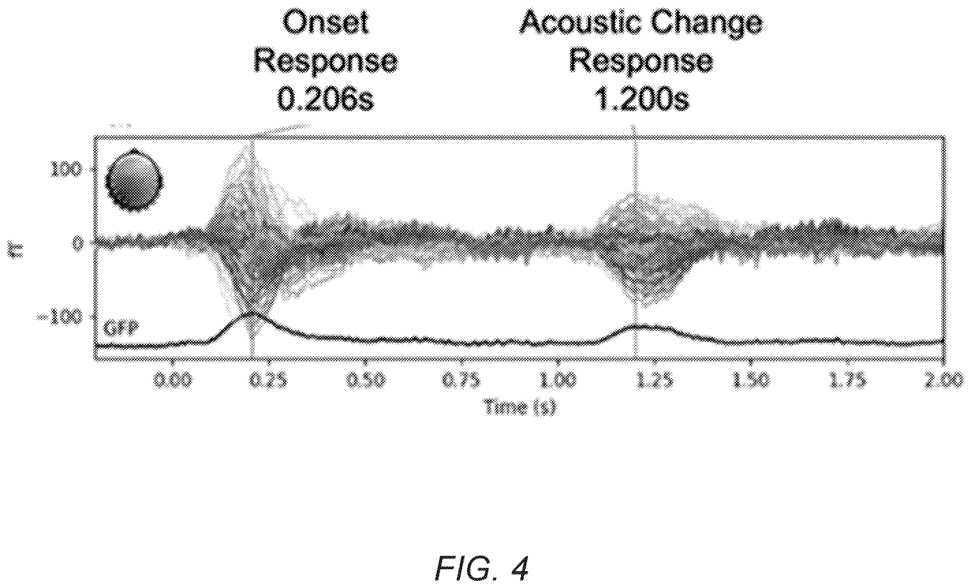

A comparison of onset and acoustic change responses to speech is illustrated in FIG. 4. The measurements depicted are averaged across 13 6-month-olds. The ACR is a well-studied response in adults and is elicited by an acoustic change in a sound. Stimuli configured in accordance with some embodiments of the invention may elicit ACRs to changes in syllables. The amplitude and/or latency of ACRs can, additionally or alternatively, be extracted and analyzed in the same way as the latency of the onset response. For example, FIG. 4 illustrates response latencies marked for both onset (0.206 s) and ACR (1.200 s) measurements. Moreover, as there are no differences expected between onset and ACR latencies extracted from MEG versus EEG recordings of AEPs, the two modalities may be used interchangeably.

ii. Cortical Tracking

Cortical tracking of the speech envelope correlates well with (e.g., behavioral measures of) speech perception and describes the synchronization between the speech envelope (slow amplitude fluctuations of the speech signal over time) and neural activity in auditory regions. LLE detection can utilize neurological recordings to analyze the speech envelope with more naturalistic, continuous speech stimuli. In certain embodiments, feature models derived from such analyses may evaluate various features, such as (but not limited to) phonetic features and/or acoustic features.

As mentioned above, cortical processing measures may prioritize real-world relevance by presenting naturalistic, continuous, infant directed speech (IDS), as opposed to adult directed speech, to ensure the most optimal neural response. In doing so, infants may listen to speakers talking to them in IDS, similar to what they may encounter in their everyday environment. This difference is motivated by the fact that an additional significant benefit of naturalistic stimuli is a dramatic improvement in participant attention and engagement during recording with continuous speech as opposed to repeated stimuli. This approach drastically increases: the success rate of obtained EEG recordings, the signal-to-noise (SNR) ratio of the obtained EEG response, and the capacity to maintain the infants' attention throughout the recording.

In accordance with some embodiments, target stimuli may be made up of naturally recorded IDS produced by native speakers (e.g., of English). The stimulus recording may take place in a sound-attenuated booth, with each talker producing a recording of a set duration (e.g., ~30 min). Stimuli may be inspected for artifacts and/or (e.g., root mean square/RMS) normalized. In some cases, EEG data can be recorded at a set sampling rate (e.g., 2,048 Hz) with values of the speaker(s) being set, including but not limited to volume (e.g. at an overall level of 70 dB SPL), distance (e.g., 25 cm), alignment (e.g., 0° azimuth), number of speakers (e.g., 2), and/or genders of speakers (e.g., female) across recordings. Additionally or alternatively, IDS segments of set duration and/or quantity (e.g., 12 clips, each with recording length of 1 min) may be presented to each participant, in quiet or embedded in noise (e.g., multi-talker babble).

In accordance with various embodiments of the invention, analyses of high-level cortical processing may be implemented using modeling configurations including but not limited to the temporal response function (TRF) method (e.g., using a combination of linear modeling and machine learning). In some cases, a multivariate variation of the TRF method (mTRF) may be implemented. The mTRF approach may be used to compute the relationship between how well the input stimulus is reflected in the recorded EEG signal using linear modelling.

LLE detection in accordance with a variety of embodiments of the invention may, additionally or alternatively, implement a backward modelling approach that decodes (or reconstructs) the amplitude envelope of the speech stimuli (“stimulus envelope”) based on recorded neural activity. Decoding models implemented in accordance with various embodiments of the invention may integrate information from some or all EEG channels. When all EEG channels are used, decoding models may assign lower weight to irrelevant channels. Decoding models may be more robust against signal-to-noise ratio (SNR) variability, which is characteristic of infant EEG data. The backward models (i.e., decoder), g(τ, n), may represent the linear mapping from the recorded neural response, r(t+n), back to the stimulus s(t), where t represents the time and n represents the EEG channel. In such cases, the reconstructed stimulus envelope, ŝ(t), may be expressed as:

$$\hat{s} ( t ) = {\sum }_{n} {\sum }_{τ} r ( t + τ , n ) g ( τ , n ) .$$

Decoders can integrate the neural response over a specified range of time lags, t, that will be optimized for envelope reconstruction.

A process for reconstruction of stimulus envelopes in accordance with certain embodiments of the invention is illustrated in FIG. 5. Reconstruction processes may be implemented based on recorded neurological signals. In some embodiments, decoding models used for reconstruction may include machine learning models (e.g., encoder-decoder models, ridge regression, other linear-regression models, etc.).

Process 500 performs (520) subject-specific decoding on a recorded neurological signal to reconstruct the stimulus envelope. Reconstructed stimulus envelopes predict the stimulus envelope that resulted in a particular neurological signal. Stimulus envelopes in accordance with a number of embodiments of the invention include an amplitude envelope of the recorded neurological signal in response to stimuli signals. Stimuli envelopes can include an amplitude envelope of the stimuli. Processes in accordance with a variety of embodiments of the invention extract the stimulus envelope within a certain frequency (e.g., between 1-8 Hz). In several embodiments, the extraction may be performed using harmonic functions including but not limited to Hilbert transform. In some cases, the extracted stimulus envelope may be down sampled (e.g., to 128 Hz) to match the neurological data.

In miscellaneous embodiments, subject-specific decoding may be performed based on a trained machine learning model. Machine learning models in accordance with a variety of embodiments of the invention may be trained to decode a stimulus envelope to reconstruct the stimulus envelope. Models in accordance with various embodiments of the invention are trained on data for the individual being evaluated. In many embodiments, models may be trained on m−1 trials, where m represents a total number of trials in a given training dataset, with each trial rotated m−1 times, serving once as the test set with the remaining trials serving as the training set. The models tested may be obtained by averaging over the single-trial models assigned to each training set.

Model parameters in accordance with some embodiments of the invention may include, but are not limited to, determinations of regularization parameters (2) and/or time lag parameters (τ). The parameter search for the regularization parameter may be implemented to optimize model performance, which can improve the quality of fit for noisy data and limits overfitting. As mentioned above, the time-lag window may be determined to optimize envelope reconstruction. In various embodiments, systems may adopt a time-lag window between 0 and 500 ms to be considered for the model fit, which has been found to be an optimal window to capture infant neural responses.

Process 500 evaluates (530) reconstruction success for the reconstructed stimulus envelope compared to the stimulus envelope from the stimuli. In a number of embodiments, reconstruction success is based on reconstruction accuracy and chance accuracy. Reconstruction accuracy in accordance with certain embodiments of the invention may be measured by calculating Pearson's correlation coefficient, r, between the reconstructed envelope and the original stimulus. To determine whether correlations obtained are greater than chance, a random permutation procedure can be conducted to produce a “chance r”. To obtain a random permutation of the data, the stimulus and recorded EEG may be offset by one trial, thus creating a mismatch between the stimulus and EEG signal. When a participant's reconstruction accuracy is greater than the chance accuracy, it can be asserted that the reconstruction of the stimulus envelope was successful. In various embodiments, reconstruction success is used as a high-level neurological measurement.

While specific processes for reconstruction of stimulus envelopes are described above, the above data analysis steps can be applied to any of a variety of processes as appropriate to the requirements of specific applications. In some embodiments, steps may be executed or performed in any order or sequence not limited to the order and sequence shown and described. In numerous embodiments, some of the above steps may be executed or performed substantially simultaneously where appropriate or in parallel to reduce latency and processing times. In miscellaneous embodiments, one or more of the above steps may be omitted.

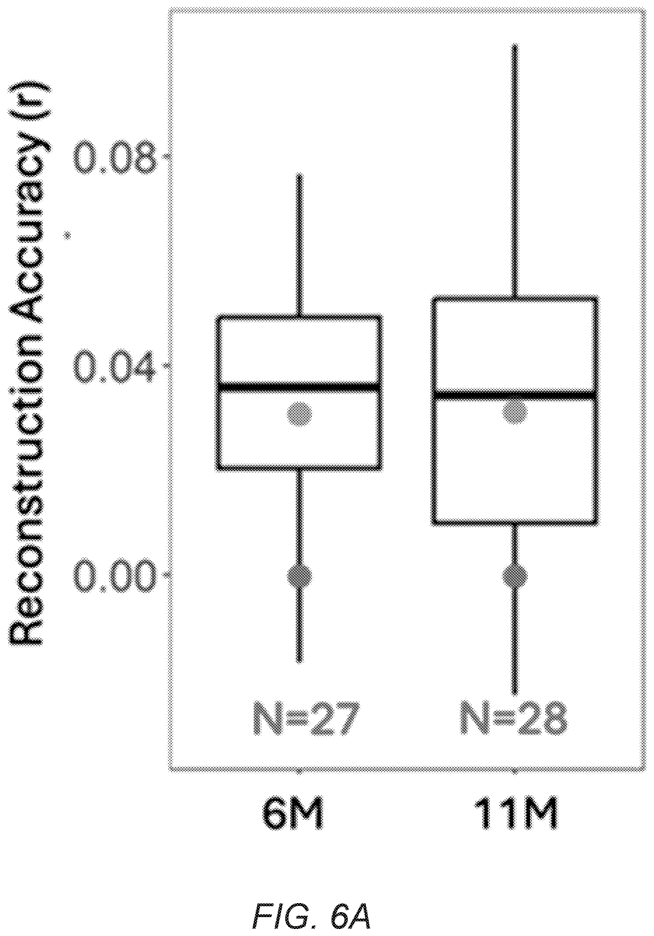

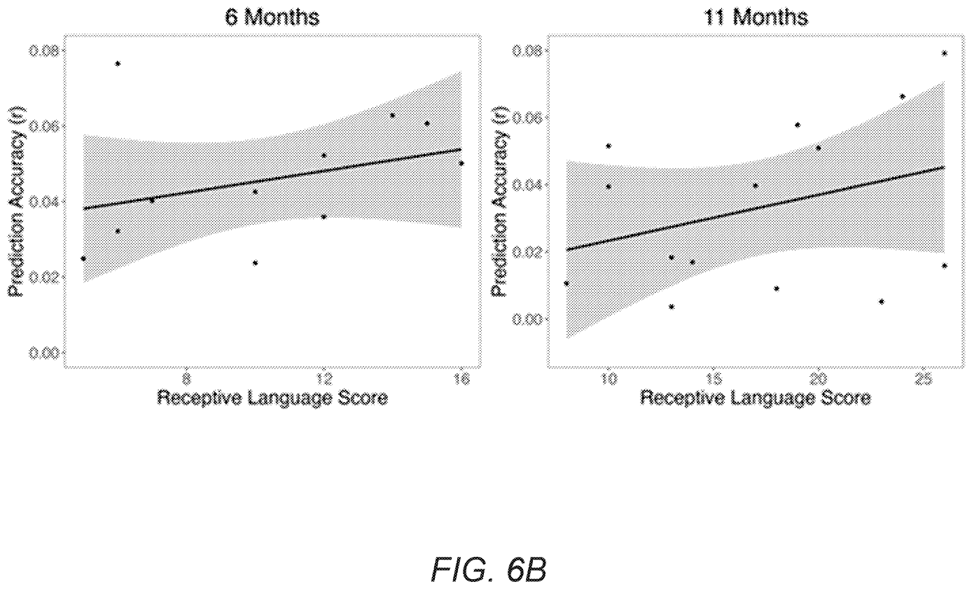

Representations evaluating the accuracy of speech envelopes reconstructed in accordance with some embodiments of the invention are illustrated in FIGS. 6A-6B. Both sets of representations compare the estimates determined for 6-month-olds and 11-month-olds tested cross-sectionally. Specifically, the graphs depicted are derived from obtained preliminary data from an additional 27 6-month-olds and 28 11-month-olds. Systems implemented in accordance with some embodiments of the invention were successful at reconstructing the speech envelope in about 90% of infants tested. In FIG. 6A, the lower dots on each graph represent chance correlation, while the upper dots represent age group mean. Moreover, the scatterplots of FIG. 6B, compare speech envelope reconstruction accuracy with receptive language abilities (measured by the Vineland Adaptive Behavior Scales-Third Edition). Both sets of graphs suggest higher speech envelope decoding accuracy in associated with infants with higher receptive language abilities. Therefore, as suggested above, in accordance with many embodiments of the invention, cortical encoding of the speech envelope at earlier points in infant development (e.g., 3 months, 6 months, 11 months) may be a predictor of language outcomes at later points (e.g., 30 months).

In addition or alternative to the approaches to modeling discussed above, systems and methods in accordance with various embodiments of the invention may employ generic, subject-independent modelling approaches which can improve the robustness of the prediction measures in secondary analysis. Generic models can be created by averaging across the trained subject-specific models of all but one participant (n−1). For each participant, n, systems can compute the correlation, r, between the predicted response based on the n−1 generic model and/or the recorded EEG response of n. This is one reason to focus on the decoding models, as they are less affected by differences in signal-to-noise ratios (which itself is likely to have a significant impact of EEG measurements, especially for infants). In terms of reliability, use of longer trials (1 minute), means that the prediction accuracies, even when small, are invariably highly significant. In secondary analysis, systems may, additionally or alternatively explore the normalization of prediction accuracies by individual subject SNRs to compare across individuals and/or match-vs-mismatch (deep learning model) classification scores.

In addition to backward modeling approaches, systems and methods in accordance with various embodiments of the invention may implement forward modeling, also referred to as encoding model analysis. In contrast to decoding models that reconstruct the stimulus envelope from neural activity, encoding models predict neural responses from stimulus features. In accordance with several embodiments, encoding models may be based on multivariate models including features such as the envelope, envelope derivative, word onset, pitch contour, phoneme identify, semantic features, and phoneme onset. Encoding model analyses may provide an alternative approach for analyzing the relationship between stimuli and neural activity, and may be particularly useful for investigating how specific stimuli features are represented in cortical responses.

In accordance with several embodiments of the invention, EEG recordings may vary in implementation. As infant participants are not able to comply with verbal instructions, EEG recordings may be conducted with third parties (e.g., researchers) to manage the infant's arousal, behavior, and attention. Systems in accordance with various embodiments may follow a behavioral coding scheme for affect and attention: the recordings may be live coded for attention to the task and state of infants throughout. For the continuous speech measure, systems may, again, use infant directed speech as opposed to adult directed speech, as there is a large body of research that suggests that IDS better captures infant attention. In preliminary data acquisition, systems have observed that infants enjoy listening to the IDS stimuli as demonstrated by sitting still, pausing movement, and smiling with the stimuli. For both measures, when an infant appears sleepy, they may be aroused and/or the recording stopped. When the infant appears inattentive, systems may regain the infant's attention and have them sit still to facilitate listening. When regaining the infant's attention is not possible, they may be given a break and/or the recording session may end. When the infant is producing excessive motion artifacts, recordings may be paused until the subjects are calmed prior to continuing the recordings. Sleepiness during the recording session may also be assessed offline via changes in alpha in the recorded EEG data. For children with “drowsy” data, systems may determine whether those children are outliers.

In accordance with various embodiments of the invention, auditory brain responses may be reviewed and evaluated. In doing so, systems may gauge missing and/or erroneous values. These reviews may, additionally or alternatively, be used to conduct data visualization for emerging growth trajectory patterns as well as correlations among measures. For the speech evoked AEP, the latency of the onset response, and/or for the continuous speech EEG, the reconstruction accuracy of the speech envelopes may be extracted from the data as acquired.

b. Additional Factors

As mentioned above, in accordance with many embodiments of the invention, developmental factors (reflective of language capabilities) including but not limited to behavioral metrics and personal background attributes may be collected and used to predict LLE. In many cases, behavioral metrics may be used to yield substantial error variance for accurately predicting late talker status. Additionally or alternatively, accurate prediction of language outcomes may require a combination of brain and behavioral measures, that may involve incorporating the contexts of demographic and environmental variables.

Personal background attributes may be obtained for use as moderating variables and for descriptive and/or supplemental analyses. These attributes may include but are not limited to birth order, household composition, social economic status (e.g., as measured by the Hollingshead Index), intervention history, special education services, family medical history, language exposure/experience, and handedness. In many cases, personal background attributes may be obtained at the start of a given evaluation (e.g., via caregiver questionnaire).

Systems may obtain behavioral measures of development across domains longitudinally at various (e.g., 3, 6, 11, 18, 30 months) ages. Additionally or alternatively, combinations of measurement metrics (e.g., that may vary in time of implementation) may be implemented to provide thorough characterization of early auditory and language abilities. These metrics may include, but are not limited to:
(1) The Parental Stress Scale: a questionnaire assessing parents' feelings about their parenting role including both positive and negative aspects of parenthood.
(2) Communication and Symbolic Behavior Scales Infant-Toddler Checklist (CSBS): a norm-referenced screening measure to detect early delays in social communication, expressive speech and language, and symbolic function.
(3) Mullen Scales of Early Learning (Mullen): a direct behavioral assessment of gross motor, visual reception, fine motor, and receptive and expressive language skills.
(4) Vineland Adaptive Behavior Scales—Third Edition (Vineland-3): a standardized assessment of communication, daily living skills, socialization, motor, and maladaptive behaviors obtained via parent report.
(5) MacArthur-Bates Communicative Development Inventories: Words and Gestures (MB-CDI: WG): a parent-report instrument where parents document their child's understanding and production of early vocabulary words along with communicative and symbolic gestures used by their child.
(6) MacArthur-Bates Communicative Development Inventories: Words and Sentences (MB-CDI: WS): a questionnaire administered to obtain an inventory of the child's word production and early grammar at 18 and 30 months.
(7) Bayley Scales of Infant and Toddler Development: an assessment used to evaluate the developmental functioning of infants and toddlers across cognitive, language, motor, social-emotional, and adaptive behavior domains.

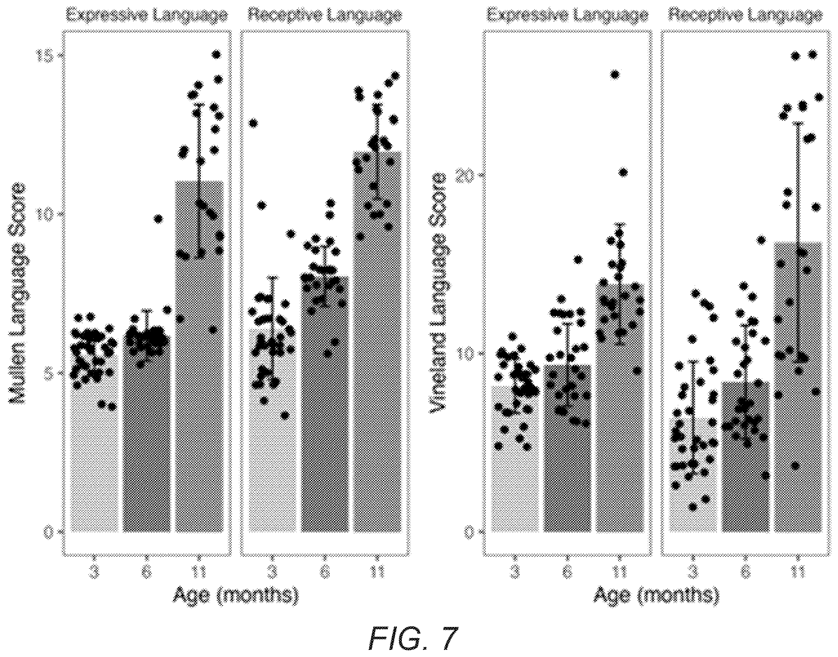

A representation of the variability of various behavioral measures, obtained for infant subjects in accordance with multiple embodiments of the invention, is depicted in FIG. 7. Specifically, the graph of FIG. 7 reflects Mullen and Vineland-3 estimates of expressive and receptive language abilities collected for infants at 3 months, 6 months, and 11 months. This preliminary data shows significant variability in the Mullen and Vineland-3 estimates at 11 months, but noticeably less variability at 3 and 6 months. This supports the conclusion that behavior-based measures can play an especially critical role in supporting the neurological (i.e., EEG) measurements and refining predictions for LLE outcomes for younger infants.

Records of language assessments may, additionally or alternatively, be used to train systems operating in accordance with various embodiments of the invention. In doing so, systems may collect comprehensive age-appropriate language assessment batteries for older infants (e.g., 30 months) to assess diagnostic accuracy. To capture the complexity of early language skills, these language assessments may include but are not limited to norm-referenced standardized language assessments, naturalistic play-based languages samples, and/or measures of visual spatial skills and working memory. As such, these assessments may allow systems to control for intellectual ability as a potential covariate to early language development. In some embodiments of the invention, specific language assessments may include but are not limited to:
(1) Clinical Evaluation of Language Fundamentals Preschool-3 (CELF-P3): Although the CELF-P3 is only normed down to 3 years of age, systems may use it to assess language and communication skills (e.g., at 30 months). An alternate option is the Preschool Language Scales—Fifth Edition (PLS-5)—however, this scale is known to be a less challenging assessment and thus, less sensitive at identifying children with mild-to-moderate language difficulties. Systems may opt to use the raw scores of (e.g., the CELF-P3) more challenging assessments to have more sensitive methods for identifying the full spectrum of late talkers. In some cases, the cutoff for late talking may be predetermined—(e.g., based on the score of the lowest 20th percent).
(2) The Eliciting Language Samples for Analysis-Toddler version (ELSA-T)—The ELSA-T uses naturalistic play-based interactions to elicit expressive language samples from toddlers. Systems may use the ELSA-T to help standardize elicitations across participants. In prior projects looking at narrative language samples from neurodiverse individuals, natural language samples were effective in capturing subtle qualitative language differences not captured by standardized assessments, especially when the participant sample is heterogenous. Thus, the language sample may be a critical complement to our battery of standardized assessments, parent questionnaires, and brain measures. The variables extracted from the obtained samples may include but are not limited to: mean length of utterance in morphemes (MLU-M), word types, and the Index of Productive Syntax (IPSyn).
(3) Wechsler Preschool and Primary Scales of Intelligence-Fourth Edition (WPPSI-IV)—As intellectual ability is a potential covariate to early language acquisition, subsets of the WPPSI-IV: Block Design, a measure of visual spatial skills and picture memory, may be administered a measure of working memory as a proxy of general intellectual ability.

In addition or alternative to the above direct assessments, systems may be trained on other behavioral (e.g., parent-reported) measures and/or personal background attributes. LLE detection processes may be based over values determined over the course of continuous processes in accordance with some embodiments of the invention. One test trial, used in training systems in accordance with many embodiments, obtains measures of auditory and linguistic abilities at five timepoints: 3, 6, 11, 18, and 30 months. Further, systems may account for infants from high likelihood (HL) categories for LLE, including but not limited to (1) prematurity and/or low birth weight, (2) having a sibling and/or parent with autism, and (3) having a sibling and/or parent with a communication disorder(s).

c. Diagnosis

Systems in accordance with many embodiments of the invention may be configured to estimate mean growth trajectories for measures including but not limited to infant EEG data, and Vineland-3 and/or Mullen language data. These estimates may be performed using multilevel linear regression modeling (also known as random/mixed effects modeling). This may be done by graphically visualizing the data to (1) ensure that the data does not exhibit any anomalies and (2) determine whether the form of linear modelling (e.g., piecewise, polynomial) that would best fit the data. Systems may, additionally or alternatively, check distributional properties to determine whether transformations (and/or other generalized linear models) are appropriate. In particular, the EEG data from pilot studies indicates normal distributions.

Systems may, additionally or alternatively, explore the correlations among intercepts and growth rates. This may be done through processes including but not limited to extracting infant-level, model-implied intercepts and rates of change; and/or evaluating the correlations among these values for annual analyses of partial data (e.g., for the relevant time period). Once data collection is complete, systems can employ parallel latent growth modeling within structural equation modeling frameworks (e.g., in Mplus) to assess the relationships simultaneously (i.e., three latent trajectories in which intercepts and slopes are freely correlated). Results from these analyses will shed light on unique relationships, such as those between changes over time in EEG data and changes over time in behavioral development measures.

Finally, systems may use growth trajectory model results to predict clinically relevant language outcomes assessed for older (e.g., 18 months, 30 months) using metrics including but not limited to the CDI, CELF, and the ELSA-T, designed to measure observed late-talker status. In doing so, systems may examine correlations with early language outcomes as measured at earlier timepoints (e.g., 6 months, 11 months, 18 months) by one or more behavioral measures (e.g., Communication and Symbolic Behavior Scales). Again, for preliminary analyses, systems may extract the model-implied intercepts and rates of change from the initial models to predict language outcomes. Additionally or alternatively, systems may control for covariates including but not limited to sex, date of assessment, and/or initial risk status (low likelihood vs. high likelihood). In some cases, systems can build upon the parallel growth modeling described above to predict these final outcomes in simultaneous models by predicting time-specific latent variables (for example, one model predicting language at 18 months and one at 30 months). In various embodiments, power analyses may be conducted for each of the relevant models. In some embodiments, these analyses may use Mplus model-based Monte Carlo utility with (e.g., 1000) replications.

Although specific implementations of LLE-predictive processes are illustrated above with respect to FIGS. 1-7, any of a variety of elements can be utilized to assess learning development similar to those described herein as appropriate to the requirements of specific applications. Additionally or alternatively, although specific methods of evaluating speech capabilities are discussed above with respect to EEG signals, other signal recording modalities can be incorporated such as (but not limited to) intracortical neural signals, EMG signals, ECoG signals, and/or any other signals as appropriate to the requirements of specific applications of embodiments of the invention.

### B. Systems for LLE Detection

a. Speech Assessment System

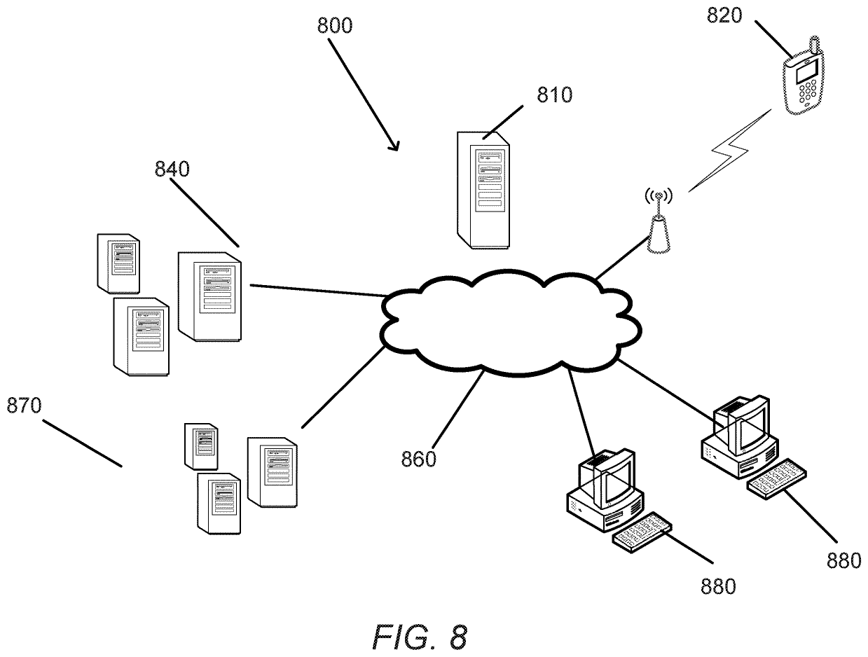

An example of a speech assessment system that evaluates likelihood of LLE in accordance with many embodiments of the invention is illustrated in FIG. 8. The speech assessment system 800 includes a communications network 860. The communications network 860 is a network such as the Internet that allows devices connected to the network 860 to communicate with other connected devices. Server systems 810, 840, and 870 are connected to the network 860. Each of the server systems 810, 840, and 870 is a group of one or more servers communicatively connected to one another via internal networks that execute processes that provide cloud services to users over the network 860. One skilled in the art will recognize that a system may exclude certain components and/or include other components that are omitted for brevity without departing from this invention.

For purposes of this discussion, cloud services are one or more applications that are executed by one or more server systems to provide data and/or executable applications to devices over a network. The server systems 810, 840, and 870 are shown each having three servers in the internal network. However, the server systems 810, 840 and 870 may include any number of servers and any additional number of server systems may be connected to the network 860 to provide cloud services. In accordance with various embodiments of this invention, a speech assessment system 800 that uses systems and methods that evaluate potential for LLE in accordance with various embodiments of the invention may be provided by a process being executed on a single server system and/or a group of server systems communicating over network 860.

Users may use personal devices 880 and/or mobile devices 820 that connect to the network 860 to perform processes that evaluate speech and/or attempts at speech in accordance with various embodiments of the invention. In the shown embodiment, the personal devices 880 are shown as desktop computers that are connected via a conventional “wired” connection to the network 860. However, the personal device 880 may be a desktop computer, a laptop computer, a smart television, a handheld EEG, or any other device that connects to the network 860 via a “wired” connection. The mobile device 820 connects to network 860 using a wireless connection. A wireless connection is a connection that uses Radio Frequency (RF) signals, Infrared signals, or any other form of wireless signaling to connect to the network 860. In the example of this figure, the mobile device 820 is a mobile telephone. However, mobile device 820 may be a mobile phone, Personal Digital Assistant (PDA), a tablet, a smartphone, or any other type of device that connects to network 860 via wireless connection without departing from this invention.

As can readily be appreciated the specific computing system used for diagnostics is largely dependent upon the requirements of a given application and should not be considered as limited to any specific computing system(s) implementation.

b. Predictive Elements

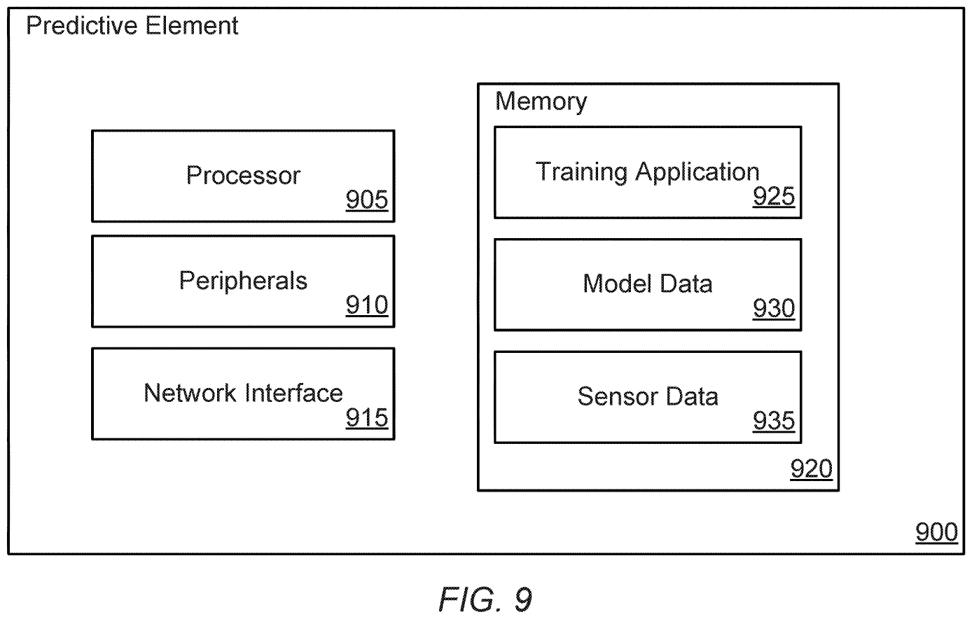

An example of a learning-directed element that executes instructions to perform processes for generating and/or training predictive models (including but not limited to machine learning models) in accordance with certain embodiments of the invention is illustrated in FIG. 9. Predictive elements 900 can include (but are not limited to) one or more of mobile devices, cameras, and/or computers. Predictive elements 900 may include but are not limited to at least one processor 905, peripherals 910, a network interface 915, and memory 920. One skilled in the art will recognize that predictive elements 900 may exclude certain components and/or include other components that are omitted for brevity without departing from this invention.

The (at least one) processor 905 can include (but is not limited to) a processor, microprocessor, controller, or a combination of processors, microprocessor, and/or controllers that perform instructions stored in the memory 920 to manipulate data stored in the memory 920. Processor instructions can configure the processor(s) 905 to perform processes in accordance with certain embodiments of the invention. In various embodiments, processor instructions can be stored on a non-transitory computer-readable and/or machine-readable medium.

Peripherals 910 can include any of a variety of components for capturing speech-related and/or neurological data used in deriving models, such as (but not limited to) cameras, displays, brain sensors, microphones, and/or other sensors. In a variety of embodiments, peripherals can be used to gather inputs and/or provide outputs of various users that can be stored as sensor data 935. Predictive elements 900 can utilize network interfaces 915 to transmit and receive data over a network based upon the instructions performed by processor(s) 905. Peripherals and/or network interfaces in accordance with many embodiments of the invention can be used to gather inputs that can be used to operate systems and/or processes including but not limited to onset response latency measurements and high-level cortical processing measurements.

Memory 920 may include but is not limited to a training application 925, model data 930, and sensor data 935. Sensor data 935 in accordance with a variety of embodiments of the invention can include EEG signals that can be used in evaluation and/or training processes. In certain embodiments, sensor data 935 can include (but is not limited to) video, images, audio, brain scans, etc. In several embodiments, model data 930 can store various parameters and/or weights for various models that can be used for various processes as described in this specification. Model data 930, in accordance with many embodiments of the invention, can be updated through training performed by the training application 925. Training may be performed based on sensor data 935 captured and/or updated (e.g., remotely) in the predictive element 900. The training application 925 can (additionally or alternatively) be used to perform methods including but not limited to sensor data 935 analytics and model updating.

Although a specific example of a speech assessment system is illustrated in FIGS. 8-9, any of a variety of elements can be utilized to evaluate infant learning trajectory similar to those described herein as appropriate to the requirements of specific applications. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for assessing language development, the method comprising:
obtaining a set of low-level neurological measurements based on a first set of stimuli, wherein the set of low-level neurological measurements comprises an onset response latency measurement;
obtaining a set of high-level neurological measurements based on a second set of stimuli, wherein the set of high-level neurological measurements are based on a temporal response function (TRF) method;
obtaining a set of developmental measurements based on at least a set of behavioral screening assessments; and
computing a predicted developmental trajectory based on a combination of the set of low-level neurological measurements, the set of high-level neurological measurements, and the set of developmental measurements.

2. The method of claim 1, wherein the first set of stimuli comprises stimuli with a characteristic selected from the group consisting of a long duration, a long interstimulus interval (ISI), and an increased sound level to ensure the most optimal neural response.

3. The method of claim 1, wherein the set of low-level neurological measurements comprises at least one selected from the group consisting of onset response latency, onset response amplitude, acoustic change response (ACR) latency, ACR amplitude, mismatch negativity, and hemispheric laterality.

4. The method of claim 1, wherein the set of high-level neurological measurements are based on a MTF.

5. The method of claim 4, wherein obtaining the set of high-level neurological measurements comprises:
decoding neurological signals to reconstruct a stimulus envelope using a trained machine learning model; and
evaluating the reconstruction success for the stimulus envelope.

6. The method of claim 5, wherein the set of low-level neurological measurements, the set of high-level neurological measurements, and the set of developmental measurements are for a particular subject, wherein the machine learning model is trained on data for the particular subject.

7. The method of claim 1, wherein the first and second sets of stimuli comprise at least one selected from the group consisting of speech stimuli and visual stimuli.

8. The method of claim 1, wherein the set of behavioral screening assessments comprises at least one selected from the group consisting of the Parental Stress Scale, the Communication and Symbolic Behavior Scales Infant-Toddler Checklist, the Mullen Scales of Early Learning, the Vineland Adaptive Behavior Scales, the MacArthur-Bates Communicative Development Inventories: Words and Gestures, the MacArthur-Bates Communicative Development Inventories: Words and Sentences, and the Bayley Scales of Infant and Toddler Development.

9. The method of claim 1, wherein at least one of the first set of neurological measurements or the second set of neurological measurements is based on an electroencephalogram (EEG) response to the first or second sets of stimuli.

10. The method of claim 1, wherein the first set of neurological measurements comprise auditory evoked potentials.

11. The method of claim 1, wherein the predicted developmental trajectory is derived from a mean growth trajectory determined based on multilevel linear regression modeling from a large sample of infants.

12. A non-transitory machine readable medium containing program instructions that are executable by a set of one or more processors to perform a method comprising:
obtaining a set of low-level neurological measurements based on a first set of stimuli, wherein the set of low-level neurological measurements comprises an onset response latency measurement;
obtaining a set of high-level neurological measurements based on a second set of stimuli, wherein the set of high-level neurological measurements are based on a temporal response function (TRF) method;
obtaining a set of developmental measurements based on at least a set of behavioral screening assessments; and
computing a predicted developmental trajectory based on a combination of the set of low-level neurological measurements, the set of high-level neurological measurements, and the set of developmental measurements.

13. The non-transitory machine readable medium of claim 12, wherein the first set of stimuli comprises stimuli with a characteristic selected from the group consisting of a long duration, a long interstimulus interval (ISI), and an increased sound level to ensure the most optimal neural response.

14. The non-transitory machine readable medium of claim 12, wherein the set of low-level neurological measurements comprises at least one selected from the group consisting of onset response latency, onset response amplitude, acoustic change response (ACR) latency, ACR amplitude, mismatch negativity, and hemispheric laterality.

15. The non-transitory machine readable medium of claim 12, wherein obtaining the set of high-level neurological measurements comprises:
decoding neurological signals to reconstruct a stimulus envelope using a trained machine learning model; and
evaluating the reconstruction success for the stimulus envelope.

16. The non-transitory machine readable medium of claim 15, wherein the set of low-level neurological measurements, the set of high-level neurological measurements, and the set of developmental measurements are for a particular subject, wherein the machine learning model is trained on data for the particular subject.

17. The non-transitory machine readable medium of claim 12, wherein the first and second sets of stimuli comprise at least one selected from the group consisting of speech stimuli and visual stimuli.

18. The non-transitory machine readable medium of claim 12, wherein at least one of the first set of neurological measurements or the second set of neurological measurements is based on an electroencephalogram (EEG) response to the first or second sets of stimuli.

19. The non-transitory machine readable medium of claim 12, wherein the predicted developmental trajectory is derived from a mean growth trajectory determined based on multilevel linear regression modeling from a large sample of infants.

20. A system for assessing language development, the system comprising:
a processor; and
a non-transitory machine readable medium containing program instructions that are executable by a set of one or more processors to perform a method comprising:
obtaining a set of low-level neurological measurements based on a first set of stimuli, wherein the set of low-level neurological measurements comprises an onset response latency measurement;
obtaining a set of high-level neurological measurements based on a second set of stimuli, wherein the set of high-level neurological measurements are based on a temporal response function (TRF) method;
obtaining a set of developmental measurements based on at least a set of behavioral screening assessments; and
computing a predicted developmental trajectory based on a combination of the set of low-level neurological measurements, the set of high-level neurological measurements, and the set of developmental measurements.
